(19) United States
(12) Patent Application Publication
(10) Pub. No.: US 2021/0096128 A1
(43) Pub. Date: Apr. 1, 2021

(54) DETERMINATION OF PROTEIN CONCENTRATION IN A FLUID

(71) Applicant: Repligen Corporation, Waltham, MA (US)

(72) Inventors: James Ronald Peyser, Billerica, MA (US); Ramsey Shanbaky, Bridgewater Township, NJ (US)

(73) Assignee: Repligen Corporation, Waltham, MA (US)

(21) Appl. No.: 17/060,546

(22) Filed: Oct. 1, 2020

Related U.S. Application Data
(60) Provisional application No. 62/909,004, filed on Oct. 1, 2019.

(51) Int. Cl. G01N 33/566; G01N 30/74; G01N 30/86; G01N 30/28

(52) U.S. Cl. CPC G01N 33/566; G01N 30/74; G01N 2030/027; G01N 30/28; G01N 30/8627

(57) ABSTRACT

The present disclosure provides systems and methods that allow users to quickly determine titer and remove hold steps by determining a first concentration using slope spectroscopy, depleting the fluid of the expressed protein by selective adsorption, and determining a second concentration using slope spectroscopy. Further, the systems and methods of the present disclosure allows the user to forgo the use of a bioanalyzer or HPLC.

### CROSS-REFERENCE TO RELATED APPLICATIONS

This application claims the benefit of priority to U.S. Provisional Application Ser. No. 62/909,004, filed Oct. 1, 2019. This application is expressly incorporated by reference herein in its entirety and for all purposes.

### FIELD

The present disclosure relates generally to a method of determining a concentration of expressed protein in a fluid.

### BACKGROUND

Monitoring and control of manufacturing processes is important in all industries, but especially in the case of biotechnology processes. Biotechnology processes are used to produce a large variety of products such as proteins, cells, tissues, carbohydrates and vaccines. Monitoring of the manufacturing process may be accomplished by in situ analysis, off line monitoring or online monitoring. Biomanufacturing processes are time consuming and usually performed in a batch mode. Continuous processing for each individual chromatography step let alone the entire manufacturing process has not yet been accomplished in a commercial process. This is partially due to the fact that samples would need to be taken at each stage of the process and then analyzed in order to ensure the efficiency of the process. The analysis of these samples would provide information that would enable the adjustment of process parameters between each step of the manufacturing process.

Currently, many of the sensors required to control the manufacturing process of biologics are available and accurate except the UV absorbance sensor (at 280 nm). Even at very small path lengths the UV sensors become saturated during the process thereby providing process operators with little or no information. This means process decisions that are made by the UV absorbance trace can only be done in the linear region of the sensor.

To circumvent this problem a surrogate wavelength of light such as 300 nm may be used, and in all cases a large effort is taken in the process development stage of a molecule to characterize this process such that a reading at 280 nm is not necessary at larger scales. This does not guarantee that problems may not occur during the process, and in fact the A300 values are not necessarily indicative of the A280 values. With batch costs in the millions of dollars, a lost batch due to a bad sensor is an unacceptable risk. During process development samples need to be taken across 30+ points at each stage of the process and analyzed for UV absorbance among other things. This step needs to be repeated many times for a single molecule at each phase of the development process.

Purification development accounts for 1-3% of the total R&D costs of a biomolecule development. For the top 12 companies, this amounts to approximately $750M -$2.2B in development costs per drug. Reducing the process development timeline, even slightly, would be of great value to industry. A direct measurement at 280 nm of the protein concentration throughout the process can reduce sampling time significantly, allow for continuous processing schemes and potentially indicate product purity during all stages of the process.

Therefore, there exists a need for a method to determine the presence of particular substances in a complex mixture of compounds simply and without purification, modification or dilution.

Spectroscopic analysis is a broad field in which the composition and properties of a material in any phase, gas, liquid, solid, are determined from the electromagnetic spectra arising from the interaction (e.g., absorption, luminescence, or emission) with energy. One aspect of spectrochemical analysis, known as spectroscopy, involves interaction of radiant energy with the material of interest. The particular methods used to study such matter-radiation interactions define many sub-fields of spectroscopy. One field in particular is known as absorption spectroscopy, in which the optical absorption spectra of liquid substances are measured. The absorption spectra is the distribution of light attenuation (due to absorbance) as a function of light wavelength. In a simple spectrophotometer the sample substance which is to be studied is placed in a transparent container, also known as a cuvette or sample cell. Electromagnetic radiation (light) of a known wavelength, λ, (ie. ultraviolet, infrared, visible, etc.) and intensity I is incident on one side of the cuvette. A detector, which measures the intensity of the exiting light, is placed on the opposite side of the cuvette. The length that the light propagates through the sample is the distance d. Most standard UV/visible spectrophotometers utilize standard cuvettes which have 1 cm path lengths and normally hold 50 to 2000 μL of sample. For a sample consisting of a single homogeneous substance with a concentration c, the light transmitted through the sample will follow a relationship know as Beer's Law: A=ϵcl where A is the absorbance (also known as the optical density (OD) of the sample at wavelength λ where OD=the −log of the ratio of transmitted light to the incident light), ϵ is the absorptivity or extinction coefficient (normally at constant at a given wavelength), c is the concentration of the sample and l is the path length of light through the sample.

Spectroscopic measurements of solutions are widely used in various fields. Often the compound of interest in solution is highly concentrated. For example, certain biological samples, such as proteins, DNA or RNA are often isolated in concentrations that fall outside the linear range of the spectrophotometer when absorbance is measured. Therefore, dilution of the sample is often required to measure an absorbance value that falls within the linear range of the instrument. Frequently multiple dilutions of the sample are required which leads to both dilution errors and the removal of the sample diluted for any downstream application. It is, therefore, desirable to take existing samples with no knowledge of the possible concentration and measure the absorption of these samples without dilution.

Multiple sample cuvettes may solve the problem of repetitive sampling, however, this approach still requires the preparation of multiple sample cuvettes and removes some sample from further use. Furthermore, in most spectrophotometers the path length, l, is fixed.

Another approach to the dilution problem is to reduce the path length in making the absorbance measurement. By reducing the measurement path length, the sample volume can be reduced. Reduction of the path length also decreases the measured absorption proportionally to the path length decrease. For example, a reduction of path length from the standard 1 cm to a path length of 0.2 mm provides a virtual fifty-fold dilution. Therefore, the absorbance of more highly concentrated samples can be measure within the linear range of the instrument if the path length of the light travelling through the sample is decreased. There are several companies that manufacture cuvettes that while maintaining the 1 cm² dimension of standard cuvettes decrease the path length through the sample by decreasing the interior volume. By decreasing the interior volume less sample is required and a more concentrated sample can be measured within the linear range of most standard spectrophotometers. While these low volume cuvettes enable the measurement of more concentrated samples the path length within these cuvettes is still fixed. If the sample concentration falls outside the linear range of the spectrophotometer the sample still may need to be diluted or another cuvette with an even smaller path length may be required before an accurate absorbance reading can be made.

The prior art also describes spectrophotometers and flow cells that are capable of measuring absorbance values of low volume samples. These devices are designed to utilize short path lengths for measuring absorbance so that only small amounts of sample are required. U.S. Pat. No. 4,643,580 to Gross et al. discloses a photometer head in which there is a housing for receiving and supporting small test volumes. A fiber optic transmitter and receiver are spaced within the housing so that a drop can be suspended between two ends.

U.S. Pat. No. 4,910,402 to McMillan discloses an apparatus in which a syringe drops liquid into the gap between two fixed fibers and an IR pulse from an LED laser is fed through the droplet. The output signal is analyzed as a function of the interaction of the radiation with the liquid of the drop.

U.S. Pat. No. 6,628,382 to Robertson describes an apparatus for performing spectrophotometric measurements on extremely small liquid samples in which a drop is held between two opposing surfaces by surface tension. The two surfaces can move relative to one another to keep the surface tension in a sample such that a spectrophotometric measurement by optical fibers can be made.

U.S. Pat. No. 6,747,740 to Leveille et al. describes a photometric measurement flow cell having measurement path lengths that can be adjusted down to less than 0.1 mm. The flow cell contains a stepped optical element which includes a stem portion that can be made to various lengths. The measurement path length can be adjusted by replacing one of the stepped elements of a particular length with another stepped element of a different length.

U.S. Pat. No. 6,188,474 to Dussault et al. describes a sample cell for use in spectroscopy that included two adjustable plates that enable a user to vary the cross sectional geometry of a sample cell flow path between two or more configurations.

U.S. Pat. No. 6,091,490 to Stellman et al. describes a fiber optic pipette coupled to a glass capillary for spectrophotometric measurements of small volume samples utilizing long path length capillary spectroscopy.

There are a series of patents assigned to Molecular Devices Corporation that describe a microplate reader capable of determining absorption measurements for multiple liquid samples in microtiter plates. Each well of the microtiter plate may provide for a different light path length based on the amount of sample solution in each well and the curvature of the meniscus of the solution in each well.

While some of these instruments provide the capability of varying the path length for measurement of highly concentrated low volume samples the applications described therein relate primarily to single path length and single wavelength measurements. Several of the instruments provide a limited number of path lengths and all are limited to path length larger than 0.2 mm. Furthermore, the devices and methods of the prior art do not provide for expanding the dynamic range of the spectrophotometer so that it is not necessary to adjust the concentration of the sample to fall within the linear range of absorbance detection of the instrument. To the extent that the prior art teaches shorter path lengths to determine the concentration of very concentrated samples or low volume samples the focus of these devices is to take a single absorbance reading at a single path length. As such the prior art references require that the path length be known with great accuracy so that an accurate concentration measurement can be made.

The present disclosure provides devices and methods that provide a variable path length spectrophotometer which dynamically adapts parameters in response to real time measurements via software control to expand the dynamic range of a conventionally spectrophotometer such that samples of almost any concentration can be measured without dilution or concentration of the original sample. Furthermore, certain methods of the present disclosure do not require that the path length be known to determine the concentration of samples. This and other objects and advantages of the disclosure, as well as additional inventive features, will be apparent from the description of the disclosure provided herein.

In order to process fluid, it may be desirable to determine the concentration of a protein before and after a processing step. It is helpful to the user for this determination step to be fast and efficient, in order to prevent significant wait times, slowing down production. Current methods such as HPLC require a column separation step and calculations from an analyst, as well as entering information such as the appropriate calibration curve and other sample information. Similarly, bioanalyzers may use enzymatic reactions that take time.

### SUMMARY

The present disclosure provides systems and methods that allow users to quickly determine titer and remove hold steps. Further, the systems and methods of the present disclosure allows the user to forgo the use of a bioanalyzer or HPLC, which is often required for these measurements. Thus, the process which would previously take between 3-10 minutes can now be performed in 2-3 minutes.

These systems and methods overcome the disadvantages and shortcomings of the prior art by providing interactive variable path length devices and methods for spectroscopic measurement of a sample. Instruments of the present disclosure can be used to measure the concentration of very concentrated samples by providing path lengths around 0.2 μm and above. Such small path lengths permit the measurement of samples too concentrated to be measured by conventional spectrophotometers. Furthermore, the instruments and methods of the present disclosure can provide spectrum scans in two or three different path length zones. This enables users to determine optimal absorbance peaks in a sample in a single run. The benefit of this method is that it can provide information on optimization of concentration measurements by comparing absorbance peak data at multiple path lengths and multiple wavelengths as these values can be different due to the contents in the sample. Instruments that use standard fixed path length cuvettes can not present all of this data at the same time. The variable path length instrument may include a probe tip, sample vessel, a mechanism for moving the probe tip and sample vessel relative to one another (e.g., the sample vessel is stationary and the probe moves or the probe is stationary and the sample vessel moves or both are capable of movement), delivery optical fiber, detector and appropriate software for path length control and measurement parameters.

The present disclosure includes methods of determining the concentration of a sample comprising placing the sample in a vessel; moving a probe relative to the vessel such that the probe makes contact with the bottom of the vessel; moving the probe relative to the vessel such that the probe moves from the bottom of the vessel through the sample by a predetermined increment such that a preselected path length through the solution is obtained; taking an absorbance reading at a predetermined wavelength; repeating steps of moving the probe relative to the sample and taking a measurement; generating a regression line from the absorbance and path length such that a slope of the regression line is obtained; determining the concentration of the sample by dividing the slope of the regression line by the extinction coefficient of the sample.

The present disclosure also includes instruments for determining the concentration of a sample at multiple path lengths comprising a light source operably linked to a probe; a sample vessel that can contain the sample; a motor operably linked to the sample vessel such that the sample vessel can be moved relative to the probe to provide variable path lengths; a probe that can carry electromagnetic radiation that can be moved relative to the sample vessel by the motor; a detector that can detect electromagnetic radiation disposed such that the detector is substantially perpendicular to the electromagnetic radiation emanating from the probe; and software that can calculate the concentration of the sample based on the information provided by the detector at the predetermined path length.

The instruments and methods of the present disclosure can be used in conjunction with a standard spectrophotometer which may be used to provide an electromagnetic source and/or a detector for measuring electromagnetic radiation. aspects and embodiments described above.

The present disclosure may relate to methods of determining the change in concentration of a substance in solution over time by continuously monitoring in real time the spectrum of the substance by detecting the spectrum in a flow through mechanism having a light source, a path length and a detector in which the path length is altered such that multiple measurements of the spectrum are made at different path lengths and then comparing the concentration of the substance over time. By having the concentration signal in real-time along with other process parameters, biomolecule mass can be determined in real-time. The present disclosure provides for monitoring the concentration of the substance at more than one location, including but not limited to at the input and at the outflow of the solution. Various substances can be monitored including but not limited to surfactants, biomolecules, proteins, cells and virus particles, small molecules, peptides or conjugated proteins.

The present disclosure may relate to methods of determining the change in concentration of a substance in solution over time by continuously monitoring in real time the spectrum of the substance where the change in concentration of the substance is due to the solution being subjected to one or more purification processes including but not limited to precipitation, filtration, electrophoresis, chromatography separation, chemical reactions and combinations thereof. Chromatographic separation may be anion exchange chromatography, cation exchange chromatography, gel filtration chromatography, hydrophobic interaction chromatography and affinity chromatography.

The present disclosure may relate to methods of determining the change in concentration of a substance in solution over time comprising monitoring in real time the ultraviolet spectrum of the substance at discrete time points by detecting the spectrum in a flow through mechanism comprising a light source, a path length and a detector in which the path length is altered such that multiple measurements of the spectrum are made at different path lengths at the discrete time points and comparing the concentration of the substance at the discrete time points. The present disclosure relates to methods of determining the lifetime of a membrane used in ultrafiltration by continuously monitoring in real time the concentration of a substance in a solution that has passed through the ultrafiltration membrane by detecting the ultraviolet spectrum of the substance in a flow through mechanism comprising a light source, a path length and a detector in which the path length is altered such that multiple measurements of the spectrum are made at different path lengths and comparing the concentration of the substance over time.

The present disclosure may relate to methods of determining the binding capacity of a chromatography resin by continuously monitoring in real time the concentration of a substance in a solution that has passed through the resin by detecting the ultraviolet spectrum of the substance in a flow through mechanism comprising a light source, a path length and a detector in which the path length is altered such that multiple measurements of the spectrum are made at different path lengths and comparing the concentration of the substance over time.

The present disclosure relates to methods of controlling a biomanufacturing process for a biomolecule by detecting a ultraviolet spectra in situ and in real-time of the biomolecule during at least one stage of the biomanufacturing process where the ultraviolet spectra characterizes a fingerprint of the biomolecule and generating at least one control signal in response to the detected ultraviolet spectra wherein the at least one signal enables a control step in the biomanufacturing process.

The present disclosure relates to methods of fingerprinting a biomolecule whereby the biomolecule spectra may be compared to a calibration curve to determine the identity of the biomolecule solution.

The present disclosure may include methods for determining a concentration of expressed protein in a fluid. This method may comprise measuring a first absorption slope value of the fluid using slope spectroscopy, and dividing said first absorption slope value by a known extinction coefficient of the protein to yield a first concentration. The fluid may be depleted of the expressed protein (e.g., by selective adsorption, precipitation, filtration, etc.), and a second absorption slope value of the depleted fluid measured using slope spectroscopy, dividing the second absorption slope value by the known extinction coefficient of the protein to yield a second concentration. Based on the first and second concentrations, an amount of material removed by the selective adsorption may be calculated. Calculating an amount of material removed may comprise subtracting the second concentration from the first concentration and multiplying the difference by a volume of the fluid. Further, measuring the first and second slope values of the fluid are may be measured at the same wavelength. The calculation of the concentration of expressed protein may use a known extinction coefficient of the expressed protein. Depleting the fluid of the expressed protein by selective adsorption may further comprise using an affinity ligand to a target protein that has been immobilized on a solid support. The target protein immobilized may be Protein A. Depleting the fluid of the expressed protein by selective adsorption may further comprise the use of one or more of the following: resin, a membrane, a filter plate, or a packed column. The resin may be dehydrated. The step of depleting the fluid may not comprise increasing a fluid volume between the first and second measurements. The step of depleting the fluid may comprise increasing a fluid volume by a predetermined amount. The steps may be automated. The fluid may be filtered prior to measurement. The fluid may require a growth period prior to measurement.

The present disclosure may include a system for determining a level of depletion of expressed protein in a fluid. This system may include a depletion module for depleting the fluid of the expressed protein by selective adsorption, first and second fluid sampling modules positioned upstream and downstream of the depletion module in a flow path of the fluid, and a slope spectroscopy apparatus configured to receive fluid from the first and second fluid sampling modules and measure an absorbance of the fluid from the sampling modules at multiple path lengths and determine a fluid concentration therefrom, wherein the depletion is determined by subtracting a concentration of the expressed protein in fluid from the second module from a concentration of the expressed protein in fluid from the first module and multiplying the difference therebetween by a volume of the fluid. The depletion module may comprise a chromatography column. The method of depleting the fluid of the expressed protein may comprise one or more of the following: immobilized Protein A, resin, a membrane, a filter plate, or a packed column. The resin may be dehydrated. The slope spectroscopy apparatus may be a slope spectrometer.

The present disclosure also provides a method of assessing the binding of an expressed protein to a chromatography column in a fluid. This method may comprise (a) taking a first absorbance spectrum of the expressed protein in the fluid at a first pathlength, (b) changing the first pathlength by an increment to provide a second pathlength and taking a second absorbance spectrum reading at the predetermined wavelength, (c) depleting the fluid of the expressed protein e.g., by selective adsorption, (d) repeating (a) and (b) on the depleted fluid, (e) generating regression lines from the absorbance values at a given wavelength such that a slope of the regression is obtained for the fluid before and after depletion of the expressed protein, (f) subtracting the slope of the depleted fluid from the non-depleted fluid slope, and (g) calculating the concentration of expressed protein. The calculation of the concentration of expressed protein may use a known extinction coefficient of the expressed protein. Depleting the fluid of the expressed protein by selective adsorption further comprises using an affinity ligand to a target protein that has been immobilized on a solid support. The target protein may be immobilized Protein A. Depleting the fluid of the expressed protein by selective adsorption may further comprise the use of one or more of the following: resin, a membrane, a filter plate, or a packed column. The resin may be dehydrated. The steps may be automated. The fluid may be filtered prior to measurement. The fluid may require a growth period prior to measurement.

### BRIEF DESCRIPTION OF THE DRAWINGS

FIG. 1 is a flow diagram of one possible embodiment of the variable path length device software set up.

FIG. 2 is a flow diagram of the data acquisition of the variable path length instrument software

FIG. 3A is a schematic of one embodiment of the instrument of the present invention.

FIG. 3B is a schematic of one embodiment of the probe tip assembly.

FIG. 4 is a schematic of a flow-through device which may serve as a sample vessel in the instruments of the present invention.

### DETAILED DESCRIPTION

### Overview

The systems and methods of this disclosure are, generally, addressed to reducing the time required for the determination of expressed protein in fluid by use of slope spectroscopy. Slope spectroscopy is a method of determining the concentration of a solution by measuring absorbance over varying pathlengths.

Those of skill in the art will appreciate that the use of slope spectroscopy for the determination of expressed protein in fluid involves several departures from existing industry standard processes. Further, it will greatly reduce the time required to process fluid.

This disclosure describes methods which measure the concentration of the input and output of bioprocesses, more specifically of the processing step. These methods may draw samples from the system before and after the processing step.

The information gathered by the methods and systems of this disclosure may be used to determine actions taken in a bioprocess. For example, a bioprocess may be delayed or accelerated due to the calculated concentration of expressed protein. Further, the information may suggest a fault or error in the bioprocessing, resulting in remedial steps being taken. Determination of the absence of protein in the fluid can trigger completion of the process, minimizing buffer usage and saving time.

Users may combine the information gathered through these methods with knowledge of the expected output volumes of permeate to determine when the expressed protein has been depleted from the fluid. Further, users may measure monoclonal antibodies by the depletion with immobilized Protein A.

The methods described may be used to assess binding capacity of a resin. They may further be used to determine efficiency of the resin, assess the sieving properties of a filter, or to determine how much material is staying on the filter rather than going through it, causing blockages.

The term “moving the probe relative to the vessel” or “moving the probe relative to the sample” means that the vessel or the sample relative to the probe is moved. This encompasses the situations where the probe is moving and the vessel or sample is stationary, the vessel or sample is moving and the probe is stationary and where the sample or the vessel is moving and the probe is moving.

The term “taking an absorbance reading” means that any absorbance reading(s) is measured by the device or instrument. This encompasses situations where the absorbance reading is taken at a single wavelength and/or a single path length or where the reading is taken at multiple wavelengths (such as in a scan) and/or multiple path lengths.

The term “sample(s)” may include, but is not limited to, compounds, mixtures, surfaces, solutions, emulsions, suspensions, cell cultures, fermentation cultures, cells, tissues, secretions, and extracts.

The term “motor” is any device that can be controlled to provide a variable path length through a sample.

The term “selective adsorption” refers to affinity-based methods for depletion of a product from a fluid. Selective adsorption may be mediated by, e.g., an affinity-binding-agent such as a peptide ligand, an antibody or functional fragment thereof, a toxin, an aptamer, a pharmacological agonist or antagonist, or other suitable reagent. The affinity binding agent is, in some cases, bound to a solid support such as a resin or particle; in other cases, the affinity binding agent is unbound, in which case depletion may be achieved by, e.g., covalent linkage of the bound affinity binding agent to a substrate via reaction of a functional group on the affinity binding agent, and/or by the use of a second binding agent such as an antibody. Other suitable affinity binding agents and depletion methods will be evident to those of skill in the art.

### Slope Spectroscopy

The present disclosure relates to devices and methods for determining the spectrophotometric characteristics of a solution by employing an approach that permits the use of a variable path length for multiple determinations of the parameters of interest. For example, in determining the concentration of a compound in solution the present disclosure provides methods and devices for determining the absorbance of the solution at various path lengths. The values of the absorbance at various path lengths can then be used to calculate the concentration of the compound in the solution. The devices and methods of the present disclosure are particularly useful for determining the concentration of highly concentrated samples without resorting to single or multiple dilutions of the samples. This attribute is possible due to the small path lengths which the devices of the present disclosure can achieve. The instruments of the present disclosure can be used to measure the concentration of very concentrated samples by providing path lengths around 0.2 μm and longer. The instruments of the present disclosure can provide path lengths from about 0.5 μm and to about 15 cm or between about 1 μm to about 50 mm. The devices and methods also provide for measurement of concentrations of extremely dilute solutions by providing larger path lengths. In essence the devices and methods of the present disclosure expand the dynamic range of a standard spectrophotometer by permitting a wide range of path lengths for measuring the absorbance values of a solution. This broad dynamic range enables users to determine the concentrations of their samples without altering (diluting or concentrating) the samples. While embodiments of the methods and devices of the present disclosure are for determining the absorbance, extinction coefficient or concentration of a particular sample or set of samples the devices and methods of the present disclosure may also be used in different modes such as scattering, luminescence, photoluminescence, photoluminescence polarization, time-resolved photoluminescence, photoluminescence life-times and chemiluminescence as well as other modalities. The devices and the methods of the present disclosure may be used to determine optical values of one or more samples at a given time. The disclosure contemplate the use of single sample formats such as cuvettes or any sample holder, as well as multiple sample formats such as microtiter plates and multiple cuvette or multiple sample arrangements.

The variable path length device of the present disclosure may be comprised of a probe tip, sample vessel, motor, delivery optical fiber, detector, unidirectional sliding mechanism and appropriate software for path length control and measurement parameters.

### Probe Tip

In the present disclosure the probe tip is a light delivery device which delivers light to the sample. The probe tip may be a single light delivery device such as a fiber optic cable that interfaces with one or more electromagnetic sources to permit passage of light through the sample. Alternatively the probe tip may be housed in a probe tip assembly which may be comprised of a light delivery device, housing, end terminations and other optical components and coatings. The light delivery device can be fused silica, glass, plastic or any transmissible material appropriate for the wavelength range of the electromagnetic source and detector. The light delivery device may be comprised of a single fiber or of multiple fibers and these fibers can be of different diameters depending on the utilization of the instrument. The fibers can be of almost any diameter but in most embodiments the fiber diameter is in the range of from about 0.005 mm to about 20.0 mm. In an embodiment the light delivery device is a single optical fiber with a diameter of from about 0.1 mm to about 1.0 mm. The probe tip optionally utilizes a housing to contain the light delivery device. This housing is used primarily to shield the light delivery device and may be made from metal, plastic, ceramic or any other material that is compatible with its usage. The probe tip may optionally include end terminations such as connectors, ferrules or anything that will facilitate a mechanical interconnection. The terminations can be polished, cleaved, shaped or manipulated in any fashion compatible with the device's usage. The instruments of the present disclosure include probe tips with additional optical components such as lenses or filters. The probe tips may include coatings on the end of the fiber tip to serve as filters, pH indicators, catalysts or as sealing mechanisms. The probe tip may be a permanent part of the instrument and/or probe assembly device or alternatively the probe tip may be detachable, such that it may be removed from the probe tip assembly. As a permanent part of the instrument the probe tip is an integral part of the light delivery device. In an embodiment the probe tip is a single optical fiber which is attached at one end to the light source and at the other end immersed in the sample. Alternatively the probe tip may be detachable and in such embodiments the probe tip can be separated from the light delivery device though a variety of mechanisms. In an embodiment the probe tip is attached to the light delivery device though a Touhey Borst adapter such that after usage the probe tip can be removed and replaced with another probe tip. The detachable probe tip is of a length sufficient to penetrate the sample and attach to the light delivery assembly. In embodiments of the detachable probe tip the length of the probe tip is at least about 20 mm in length. Depending on its usage the probe tip may simply be thrown away after removal. Disposable probe tips obviate problems associated with cleaning the probe tip and avoid the potential of contamination from one sample to another. Instruments of the present disclosure include multiple probe tips that can be associated with a single light delivery device. Alternatively multiple light delivery devices may be associated with each probe tip.

The path length is the distance between the end of the probe tip and inside surface of the sample vessel holding the liquid, the inside surface being the surface of the vessel which is substantially perpendicular to the probe tip. The end surface of the probe tip, which both defines the path length and is in contact with the liquid, is substantially parallel to the inside surface of the sample vessel which is adjacent to the detector. In one embodiment, the probe tip is positioned above the sample vessel holding the sample and aligned so that the light exiting the probe tip will pass through the sample vessel onto a detector (or detection light guide). The probe tip is able to transmit wavelengths within the range of the instrument.

### Light Source

The electromagnetic radiation source provides light in a predetermined fashion across a wide spectral range or in a narrow band. The light source may include arc lamps, incandescent lamps, fluorescent lamps, electroluminencent devices, laser, laser diodes, and light emitting diodes, as well as other sources. In an embodiment the source of radiation is a Xenon arc lamp or tungsten lamp. In an embodiment of the present disclosure the light source is coupled to the probe tip through a light guide. Alternatively the light source could be a light emitting diode that can be mounted directly onto the probe tip.

### Sample Vessel

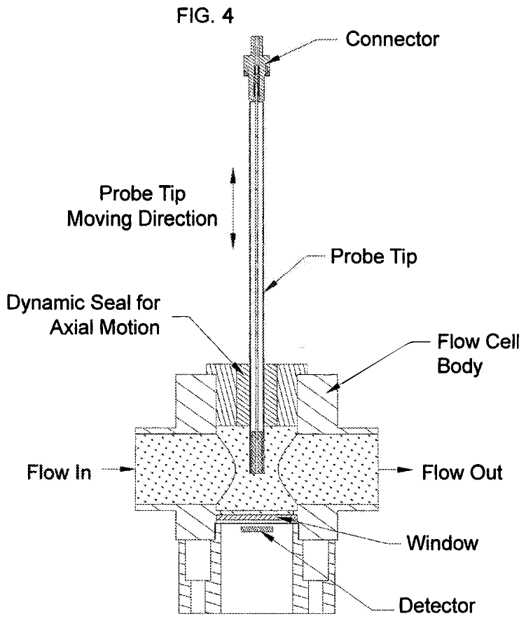

The vessel must be able to contain the liquid and allow light to pass through it onto the detection light guide or detector. The vessel will also have an opening to allow the probe tip to delivering light, to penetrate the liquid. This vessel should be able to transmit wavelengths within the range of the instrument typically from about 200-1100 nm. For ultraviolet application a quartz vessel may be required, but often plastic vessels will made of cyclo olefin polymer (COP), cyclo olefin copolymer (COC), polystyrene (PS) or polymethyl methacrylate (PMMA) will suffice. The sample vessels used with the present disclosure can be of different sizes and shapes depending upon the application and the amount of sample available for analysis. The sample vessels of the present disclosure may be anything that permits an absorbance value to be taken. Such vessels include stationary sample vessels as a cuvette or microtiter plate or moving samples as in a flow-through device (FIG. 4). The sample size may be between 0.1 μL to several liters in a stationary sample. A shape of the vessel is one with the side facing the detector being substantially flat and substantially parallel to the face of the detector. The detector may be situated at a slight angle to the vessel to reduce noise due to back reflection of the electromagnetic radiation coming through the sample. The sample vessel may have multiple wells such as in a microtiter plate. The sample vessel may be coated with optical materials or chemicals or biochemicals such as antibodies. The sample vessel may optionally be heated or cooled by the instrument and may be held in a sealed area that can be sterile or non-sterile. The sample may be held in a sample holder supported by a stage. The sample can include compounds, mixtures, surfaces, solutions, emulsions, suspensions, cell cultures, fermentation cultures, cells, tissues, secretions, extracts, etc. Analysis of the sample may involve measuring the presence, concentration or physical properties of a photoactive analyte in such a composition. Samples may refer to contents of a single well or cuvette or sample holder or may refer to multiple samples within a microtiter plate. In some embodiments the stage may be outside the instrument.

### Motor

The motor drives the tip probe into and out of the vessel. The motor drives the probe tip in precise steps to vary the path length through the sample. Path length changes can be from zero mm and larger depending upon device configuration. The motor permits the movement of the probe within the sample to place the probe tip at the precise pre-determined path length. Motors that can be used with the instruments of the present disclosure include stepper motors, servo, piezo, electric and magnetic motors or any device that can be controlled to provide a variable path length through a sample. In an embodiment of the instruments of the present disclosure the motor drives a stage on which the sample vessel rests so that the probe tip moves relative to the sample vessel. In this configuration the stage and the probe move relative to each other in increments which range from 0.2 μm to 1 cm. In an embodiment the range of increment is between from about 1 μm to about 50 μm. The relative motion of the stage to the probe is accurate to with a resolution of 0.2 μm or less. In an embodiment of the instruments of the disclosure the resolution of the relative motion of the probe and the stage is between about 0.5 μm to about 0.01 μm.

### Unidirectional Sliding Mechanism

The unidirectional sliding mechanism is a system designed to permit physical contact between the end of the probe tip and the “bottom” (perpendicular to the probe tip) of the sample vessel in order to establish a “zero path length” position which is an approximate zero benchmark from which all other path lengths can be referenced. In an embodiment of the present disclosure the unidirectional sliding mechanism insures that the probe tip makes physical contact with the sample vessel surface thereby guaranteeing that the probe tip is in the “zero path length” position. Physical contact should to be achieved without causing damage to either the sample vessel or the probe tip. In an embodiment the position is achieved by allowing/requiring linear displacement of either the sample vessel of the probe tip in one direction once the physical contact is achieved. This allows displacement in the direction that zero path length position is set, much in the same way as using the tare feature on a scale. The motion is constrained to reduce or eliminate backlash or recoil as the probe tip and vessel surface are separated. The device capable of these features is referred to as a unidirectional sliding mechanism. There are numerous embodiments of the unidirectional sliding mechanism.

In an embodiment, the unidirectional sliding mechanism comprises a modeled plastic coupling device called a Touhy Borst Adapter (TBA) which contains a silicone rubber or similarly compliant gasket material with a hole in the center of it which is housed by two threaded plastic components which when screwed together compress the internal gasket, thus reducing the diameter of the internal hole creating a seal around anything within the hole. The amount of sealing and compression can be controlled by the changing the length of threaded engagement between the two threaded components of the TBA. In an embodiment, the probe tip is inserting through the hole in the TBA gasket and then the TBA is tightened to compress the TBA gasket around the probe tip. The threading is adjusted so the frictional force between the probe tip and the TBA gasket exceeds the weight of the probe tip, thus not allowing the probe tip to fall out of the TBA when held vertically, but not so tight that the probe tip is unable to slide inside of the gasket. This frictional interaction results in a unidirectional sliding displacement that allows the establishment of the zero path length position.

There are other means and mechanisms by which this can be achieved. In one embodiment a thin membrane with a hole, a linear slit or two orthogonal slits enclosed between two blocks contains a hole slightly larger than the probe tip such that the probe tip can be inserted into the blocks and the membrane creates the frictional force that allows displacement in one direction.

In another embodiment the coupling mechanism for the probe tip or the sample vessel can comprise a spring loaded tapered sliding coupling that releases the probe tip or sample vessel when a force is applied in one direction, but grips more tightly when the force is released, similar to a spring loaded compression ring.

In another embodiment the coupling mechanism for the probe tip of the sample vessel can comprise a spring loaded ratchet mechanism which displaces a toothed slide which locks in place when displaced in one direction, but would require a release button to allow unloading or motion in the opposite direction.

In each of the embodiments of the unidirectional sliding mechanism the zero path length position is set passively, meaning the user does not need to interact with the device other than driving the motion of the system to achieve the physical contact condition. There are other embodiments that require intervention of the user, which may be utilized for long path length and flow versions of the instruments of the present disclosure. In one embodiment, the probe tip coupling mechanism has a sliding coupling. After physical contact is achieved and displacement has occurred the user will set the displacement by means of a thumb screw, a set screw, tightening a collect, mechanical clamp, magnetic clamp or other means of locking the position of either the probe tip, probe tip coupling mechanism, the sample vessel or the sample vessel holding device.

### Detector

Detectors comprise any mechanism capable of converting energy from detected light into signals that may be processed by the device. Suitable detectors include photomultiplier tubes, photodiodes, avalanche photodiodes, charge-coupled devices (CCD), and intensified CCDs, among others. Depending on the detector, light source, and assay mode such detectors may be used in a variety of detection modes including but not limited to discrete, analog, point or imaging modes. Detectors can used to measure absorbance, photoluminescence and scattering. The devices of the present disclosure may use one or more detectors although in an embodiment a single detector is used. In an embodiment a photomultiplier tube is used as the detector. The detectors of the instrument of the present disclosure can either be integrated to the instrument of can be located remotely by operably linking the detector to a light delivery device that can carry the electromagnetic radiation the travels through the sample to the detector. The light delivery device can be fused silica, glass, plastic or any transmissible material appropriate for the wavelength range of the electromagnetic source and detector. The light delivery device may be comprised of a single fiber or of multiple fibers and these fibers can be of different diameters depending on the utilization of the instrument. The fibers can be of almost any diameter but in most embodiments the fiber diameter is in the range of from about 0.005 mm to about 20.0 mm.

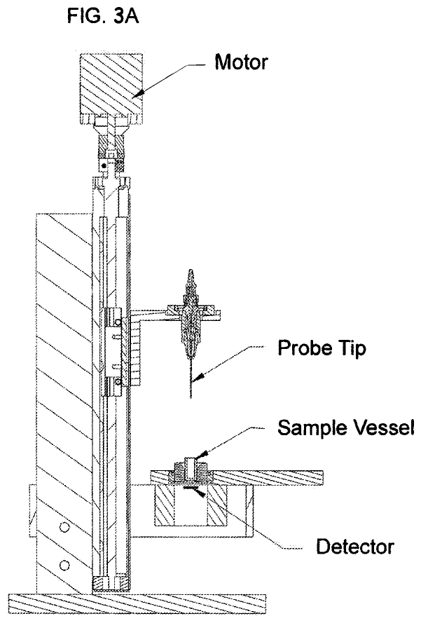

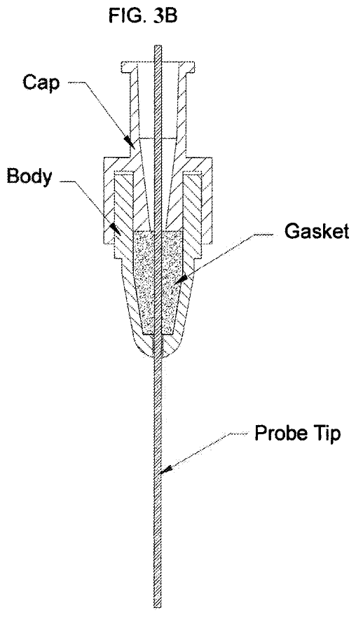

One embodiment of the instruments of the present disclosure has the optics of the system oriented such that the probe tip is on “top” and the detector is on the “bottom” (FIG. 3A). In this vertical orientation the sample vessel is above the detector and the probe tip can move up and down, into and out of the sample vessel such that the light form the probe tip moves through the sample within the sample vessel and impinges on the detector below. Other orientations are possible such as in a flow-cell system where the detector and probe tip may be in a substantially horizontal orientation (FIG. 4) and the sample flows between the detector and the probe. Regardless of the absolute spatial orientation or the probe and detector, the probe tip and surface of the detector should be substantially perpendicular relative to one another.

### Software

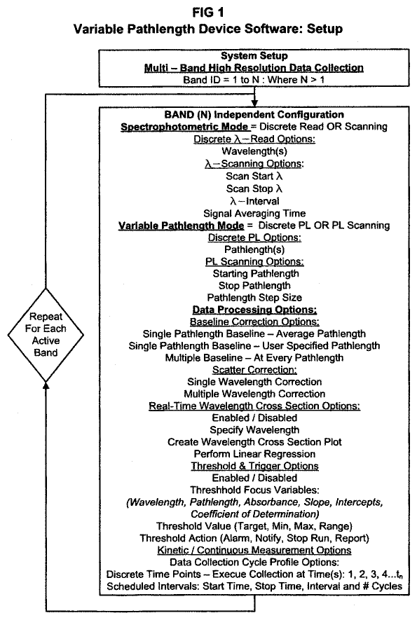

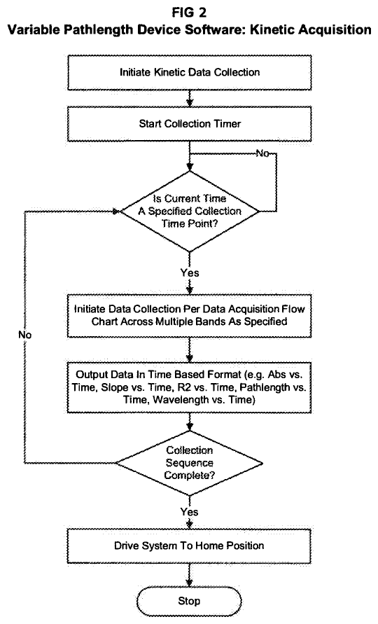

The control software will adapt the devices behavior based upon various criteria such as but not limited to wavelength, path length, data acquisition modes (for both wavelength/path length), kinetics, triggers/targets, discrete path length/wavelength bands to provide different dynamic ranges/resolutions for different areas of the spectrum, cross sectional plot to create abs/path length curves, regression algorithms and slope determination, concentration determination from slope values, extinction coefficient determination, base line correction, and scatter correction. FIG. 1 is a flow diagram of an embodiment of the software scheme of the present disclosure. The software is configured to provide scanning or discrete wavelength read options, signal averaging times, wavelength interval, scanning or discrete path length read options, data processing option such as base line correction, scatter correction, real-time wavelength cross-section, threshold options (such as wavelength, path length, absorbance, slope, intercept, coefficient of determination, etc.) an kinetic/continuous measurement options. FIGS. 2A and 2B are flow diagrams of one embodiment of the data acquisition of the variable path length instrument software. FIG. 2 is a flow diagram of one embodiment of the data acquisition of real-time data collection that can be integrated into the data acquisition program.

FIG. 3A is a schematic of one embodiment of the instruments of the present disclosure. The motor (1) drives the stage (4) on which the sample vessel (3) sits. The fiber tip probe (2) is fixed with respect to the motor such that as the stage moves up and down the probe distance to the sample vessel is increase or decreased respectively. Beneath the stage is the detector (5) which receives electromagnetic radiation from the probe tip once it has passed through the sample. FIG. 3B is a schematic of one embodiment of the probe tip assembly.

FIG. 4 is a schematic of a flow-through device which may serve as a sample vessel in the instruments of the present disclosure. The flow-through device comprises a flow cell body (8) that permits the flow of a sample solution into and out of the flow cell device. The flow cell body (8) has at least one window (7) that is transparent to electromagnetic radiation in the range of electromagnetic source typically 200-1100 nm. The window can be made from various materials but for ultraviolet applications quartz, cyclo olefin polymer (COP), cyclo olefin copolymer (COC), polystyrene (PS) or polymethyl methacrylate PMMA may be required. The window may be of different sizes and shapes so long as the electromagnetic radiation can pass through the window and strike the detector (5). The flow cell body also comprises a port through which the probe tip may pass. This port is sealed with a dynamic seal (9) such that the probe tip can pass through the port without sample solution leaking from the flow-through device. Such seals include FlexiSeal Rod and Piston Seals available from Parker Hannifin Corporation EPS Division, West Salt Lake City, Utah. In the diagram there is a single pathway for the sample solution to flow coming in the inlet port and exiting the outlet port. Alternative embodiments may include multiple pathways and multiple inlet and outlet ports. In the embodiment of the flow cell device in FIG. 4, the probe tip moves substantially perpendicular to the flow of the sample solution and is substantially perpendicular to the detector.

In one embodiment of the methods of the present disclosure multiple absorbance measurements may be taken at multiple path lengths without accurately knowing what the path length distance is. The prior art is replete with methods teaching how to accurately determine the path length in an absorbance reading so that an accurate determination of the concentration of the sample can be made. In this embodiment of the present disclosure multiple absorbance measurements made at different path lengths enables an accurate calculation of the concentration based upon the instrument's ability to calculate a regression line from the absorbance and path length information. The slope of the regression line can then be used to calculate the concentration of the sample. Each path length need not be accurately known due to the fact that the software used to calculate the regression line can be programmed to select the most accurate line from the data set presented. The number of data points taken in these methods tends to “smooth out” any perturbations in the path length or absorbance reading such that regression lines with very high R2 values can be obtained. In the methods of the present disclosure R2 values of at least 0.99999 have been achieved. Obviously the higher the R2 value the more accurate the slope which results in a highly accurate determination of the concentration of the sample. Any R2 value between 0 and 0.99999 is achievable in the instruments and methods of the present disclosure, however in some embodiments of the methods of the present disclosure the R2 value exceeds 0.95000 and in some embodiments the R2 will exceed 0.99500. In an embodiment of the present disclosure the R2 value is between about 0.95000 and about 0.99999. Other embodiments include R2 values between about 0.99500 and about 0.99999 and about 0.99990 and about 0.99999. While R2 is a measure of goodness-of-fit for the linear regression any other mathematic expression that measures goodness-of-fit can be utilized in the methods of the present disclosure.

The instruments and methods of the present disclosure allow the user to optimize the collection of data by selecting a pre-determined parameter such as absorbance. The user can define, for example, an absorbance of 1.0 and have the instrument search for other parameters (such as wavelength or path length) at which the absorbance of the sample is 1.0. This feature enables the user to define the parameters for the experiment without having to make multiple dilutions or constantly change the parameters of the instrument manually. The software of the present disclosure also permits the user to define an expected R2 value so that the level of accuracy for the outcome can be defined prior to the data acquisition.

The instruments and methods of the present disclosure permit the collection of a variety of data sets including three dimension data sets that include measurement of absorbance, path length and wavelength. The software enables the user to generate three dimensional graphs of these data sets. Furthermore, the instruments and methods of the present disclosure provide for the collection of real-time data.

The instruments and methods of the present disclosure enable the calculation of the extinction coefficient of a particular sample at different wavelengths. The extinction coefficient, also known as absorptivity, is the absorbance of a solution per unit path length and concentration at a given wavelength. If the extinction coefficient for a given sample is known at a first wavelength (ϵ1) one can calculate the extinction coefficient at a second wavelength (ϵ2). This is done by measuring the ratio of the absorbance/path length at the first wavelength (A/l)1 to the absorbance/path length at a second wavelength (A/l)2 and equating this ratio to the ratios of the extinction coefficients: (A/l)1/(A/l)2=ϵ1/ϵ2.

The instruments and methods of the present disclosure also enable the user to measure the components in a complex mixture at the same time as long as the wavelengths that identify the multiple components in the sample can be separated. For example, a conventional spectrophotometer would not in a single experiment be able to determine the concentration of a sample where there are two components A, which is highly concentrated and absorbs predominantly at 300 nm and B which is quite dilute and absorbs at 600 nm. In a conventional spectrophotometer the measurement of the absorbance due to component B would preclude the measurement of the absorbance of component A as the concentration of A is high enough as to swamp the detector. The original sample would need to be diluted to determine component A, and in doing so component B would not produce enough signal to permit its concentration to be measured. In a conventional spectrophotometer the concentration of the components A and B cannot be measured simultaneously. In the present disclosure the path length can be altered so that both the concentration of components A and B can be determined together. Obviously, as long as there are peaks which uniquely identify a component within a sample the methods of the present disclosure can measure the concentration of the components of very complex samples. Additionally because the instrument is capable of generating data in real-time, the interaction of components within the sample can be monitored to produce kinetic data or any data for which a time course is required.

Those of skill in the art may appreciate that many adsorptive methods may include the addition of fluids to the system, e.g., through the use of a wetted chromatography medium. The additional fluid may complicate the interpretations of a second adsorptive method according to the methods of this disclosure insofar as an end user may be unable to differentiate a decrease in slope or absolute absorbance due to depletion of the substance or product of interest from a decrease due to dilution of the product of interest. This is addressed in certain embodiments by reducing or eliminating any volumes of fluid that may be added to the system during the step of depletion of the fluid of the expressed protein. The step of depleting the fluid may not comprise increasing a fluid volume between the first and second measurements. A dehydrated resin may be used. This dehydrated resin may comprise Protein A. In other embodiments, the step of depleting the fluid may comprise increasing a fluid volume by a predetermined amount.

What is claimed is:

1. A method of determining a concentration of expressed protein in a fluid comprising:
measuring a first absorption slope value of the fluid using slope spectroscopy, and dividing said first absorption slope value by a known extinction coefficient of the protein to yield a first concentration;
depleting the fluid of the expressed protein by selective adsorption;
measuring a second absorption slope value of the depleted fluid using slope spectroscopy, and dividing the second absorption slope value by the known extinction coefficient of the protein to yield a second concentration; and
based on the first and second concentrations, calculating an amount of material removed by the selective adsorption.

2. The method of claim 1, wherein the step of calculating an amount of material removed comprises subtracting the second concentration from the first concentration and multiplying the difference by a volume of the fluid.

3. The method of claim 1, wherein measuring the first and second slope values of the fluid are measured at the same wavelength.

4. The method of claim 1, wherein the calculation of the concentration of expressed protein uses a known extinction coefficient of the expressed protein.

5. The method of claim 1, wherein depleting the fluid of the expressed protein by selective adsorption further comprises using an affinity ligand to a target protein that has been immobilized on a solid support.

6. The method of claim 5, wherein the target protein is immobilized Protein A.

7. The method of claim 1, wherein depleting the fluid of the expressed protein by selective adsorption further comprises the use of one or more of the following: resin, a membrane, a filter plate, or a packed column.

8. The method of claim 7, wherein the resin is dehydrated.

9. The method of claim 7, wherein the step of depleting the fluid does not comprise increasing a fluid volume between the first and second measurements.

10. The method of claim 7, wherein the step of depleting the fluid comprises increasing a fluid volume by a predetermined amount.

11. The method of claim 1, wherein the steps are automated.

12. The method of claim 1, wherein the fluid is filtered prior to measurement.

13. The method of claim 1, wherein the fluid requires a growth period prior to measurement.

14. A system for determining a level of depletion of expressed protein in a fluid comprising:
a depletion module for depleting the fluid of the expressed protein by selective adsorption;
first and second fluid sampling modules positioned upstream and downstream of the depletion module in a flow path of the fluid; and
a slope spectroscopy apparatus configured to receive fluid from the first and second fluid sampling modules and measure an absorbance of the fluid from the sampling modules at multiple path lengths and determine a fluid concentration therefrom,
wherein the depletion is determined by subtracting a concentration of the expressed protein in fluid from the second module from a concentration of the expressed protein in fluid from the first module and multiplying the difference therebetween by a volume of the fluid.

15. The system of claim 12, wherein the depletion module comprises a chromatography column.

16. The system of claim 14, wherein the method of depleting the fluid of the expressed protein comprises one or more of the following: immobilized Protein A, resin, a membrane, a filter plate, or a packed column.

17. The system of claim 14, wherein the slope spectroscopy apparatus is a slope spectrometer.

18. A method of assessing the binding of an expressed protein to a chromatography column in a fluid, comprising the steps of:
(a) taking a first absorbance spectrum of the expressed protein in the fluid at a first pathlength;
(b) changing the first pathlength by an increment to provide a second pathlength and taking a second absorbance spectrum reading at the predetermined wavelength;
(c) depleting the fluid of the expressed protein by selective adsorption;
(d) repeating steps (a) and (b) on the depleted fluid;
(e) generating regression lines from the absorbance values at a given wavelength such that a slope of the regression is obtained for the fluid before and after depletion of the expressed protein;
(f) subtracting the slope of the depleted fluid from the non-depleted fluid slope; and
(g) calculating a percentage of depletion of expressed protein by dividing the quantity calculated in (f) by the slope of the fluid before depletion.

19. The method of claim 18, wherein the calculation of the concentration of expressed protein uses a known extinction coefficient of the expressed protein.

20. The method of claim 18, wherein depleting the fluid of the expressed protein by selective adsorption further comprises using an affinity ligand to a target protein that has been immobilized on a solid support.

21. The method of claim 20, wherein the target protein is immobilized Protein A.

22. The method of claim 18, wherein depleting the fluid of the expressed protein by selective adsorption further comprises the use of one or more of the following: resin, a membrane, a filter plate, or a packed column.

23. The method of claim 22, wherein the resin is dehydrated.

24. The method of claim 18, wherein the steps are automated.

25. The method of claim 18, wherein the fluid is filtered prior to measurement.

26. The method of claim 18, wherein the fluid requires a growth period prior to measurement.
